(19) United States
(12) Patent Application Publication
(10) Pub. No.: US 2021/0196852 A1
(43) Pub. Date: Jul. 1, 2021

(54) ELECTROSPRAY IONIZATION OLFACTOMETER DEVICE, SYSTEM AND METHOD OF USE

(71) Applicant: INTERNATIONAL FLAVORS & FRAGRANCES INC., New York, NY (US)

(72) Inventors: Jeffrey Robert Kattas, Sayreville, NJ (US); Anshul Jain, East Brunswick, NJ (US); Matthias Horst Tabert, Arverne, NY (US); Timothy Young, Middletown, NJ (US)

(21) Appl. No.: 16/076,375

(22) PCT Filed: Feb. 17, 2017
(86) PCT No.: PCT/US2017/018270

§ 371 Date: Aug. 8, 2018

Related U.S. Application Data
(60) Provisional application No. 62/296,669, filed on Feb. 18, 2016.

(51) Int. Cl. A61L 9/22; A61L 9/12

(52) U.S. Cl. CPC A61L 9/22; A61L 9/127; A61B 5/4011; A61L 2209/134; A61L 9/125

(57) ABSTRACT

An electrospray ionization olfactometer device, method and system for vaporizing aroma compounds for the purpose of psychophysical experimentation and sensory evaluation are provided.

### INTRODUCTION

This application claims the benefit of priority of U.S. Provisional Application No. 62/296,669, filed Feb. 18, 2016, the content of which is incorporated herein by reference in its entirety.

### BACKGROUND

Olfactometry is the purest method for evaluating aroma compounds. However, there are certain factors that are difficult to compensate for and can make olfactometer use impractical. The majority of olfactometer designs transmit the headspace of a liquid in an enclosed vessel to a subject. The predominant issue in this approach is that every sample presented requires analytical measurement to determine the vapor phase concentration. This is often a lengthy labor-intensive process. Also, due to the nature of airflow dynamics, one cannot ensure that the vapor concentration remains constant between or during presentations. Moreover, when using mixtures of aroma compounds the different constituents may have different evaporative rates and the ratio of the mixture can change over time.

Other olfactometers have attempted to negate some of the issues by vaporizing the liquid directly at a set rate. This is accomplished by pumping a liquid into a super-heated vessel causing it to change to a gas as it is expelled from a capillary within the vessel. The benefit to this approach is that the flow of liquid is controlled so that quantity of the aroma compound in the final gaseous presentation is known beforehand and the analytical verification is no longer required. With the increased heat, however, aldehydes and other reactive species of compounds are increasingly more susceptible to oxidation and degradation thereby creating byproducts that can interfere with the intended experiment and analytical measurements. Moreover, after the compound is in the gas phase it needs to be rapidly cooled in order to be safely evaluated by a subject. This cooling can cause condensation of the aroma compound within the device. Lastly, at the flow rates required to generate low vapor concentrations, pulsatile flow can occur where the vapor concentration oscillates rather than remain constant. Therefore, an alternative olfactometer is needed.

EP 1656161 describes a method for dispensing liquid fragrances, by using a device with at least one supply line for supplying the fragrance to at least one delivery unit, wherein the supplied fragrance is converted to an aerosol by applying an electric charge, a high-voltage unit connected to the delivery unit for applying the electric charge to the fragrance, a controller, and at least one shutoff and actuating element connected with the controller for shutting off the supply line.

DE 69232096 discloses a device for generating electrostatically charged aerosols and/or vapors, wherein a porous capillary unit with several fibers transports a fragrance from a reservoir to a delivery unit. The fluid is hereby electrostatically charged by a high-voltage DC source and can be dispensed in the form of aerosols and/or vapors from the free tips of the capillary unit. The fluid is transported continuously by capillary action to the upper end of the capillary unit acting as a wick, even if no voltage is applied.

U.S. Pat. No. 6,126,086 describes an oscillating capillary nebulizer with electrospray which is capable of nebulizing a liquid sample flow at microflow and normal liquid flow rates for use in combination with bench top liquid chromatograph and mass spectrometer instrument systems and microflow separation techniques such as LC and CE combined with ICP/AES, ICP/MS, FT-IR, FT-MS and MS/MS.

U.S. Pat. No. 6,338,715 describes a digitally operated apparatus that dispenses controlled amounts of a volatile test fluid from a digital jetting device of the type used for ink jet printing, wherein the jetting device dispenses droplets of fluid onto the heater where the fluid is transformed into vapor.

U.S. Pat. No. 6,729,552 discloses a mass transfer device that disperses liquid into a vapor while substantially maintaining the liquid composition at the original composition. The device is composed of a container, a capillary device, and a housing, wherein the housing includes a first end having an opening attached thereon the container, a low voltage supplier attached to one wall, a high voltage converter attached to another wall, a voltage contact and a counter electrode.

U.S. Pat. No. 7,697,257 discloses an apparatus for generating, dispersing and delivering chemical compounds utilizing desorption electrospray ionization which includes an airflow channel with an inlet and an outlet into which an airflow is directed, a solvent reservoir containing a volume of solvent, at least one charged droplet source for producing a plurality of charged liquid droplets in the airflow channel, at least one grounded counter electrodes positioned within the airflow channel with the electrodes having at least one surface containing one or more chemical compounds that include releasable ions.

U.S. Pat. No. 7,829,847 describes a microscale electrospray emitter, which is fabricated and used to investigate an electrified air-fluid interface and the formation of quasi equilibrium states. The emitter is designed to be compatible with traditional microfluidic device fabrication and is demonstrated to be compatible with on-chip sample processing.

U.S. Pat. No. 8,973,851 discloses an apparatus using a conductive or semi-conductive fluid that moves along a solid or semisolid filament wherein upon applying a high electrical potential to the fluid, the fluid's high electrical potential relative to a second or counter electrode creates a electrical field intensity sufficient to form a stream of small and charged fluid droplets at the filament's apex as the fluid is electrically drawn towards the counter electrode.

US 2009/0314850 provides a method for atomizing active substances contained in a liquid by electrohydrodynamic means with at least one nozzle at least one electrode, wherein the nozzle and the electrode are arranged in such a way that a molecularization of the active substances contained in the liquid takes place and parasitic effects and/or disturbing influences that occur during the atomization, and in particular production of ozone, are reduced to the greatest extent.

US 2009/038646 discloses an inductively charged vapor-emitting device for dispensing a volatizable material into the surrounding environment. The inductively charged vapor-emitting device includes an inductive coil including a magnetic core mounted in a housing and coupled to at least one rechargeable power source mounted within the housing.

### SUMMARY OF THE INVENTION

This invention provides a device for vaporizing aroma compounds without heat. The device includes at least one vaporization unit, which includes at least one conductive capillary, at least one gaseous inlet, and at least one gaseous outlet; at least one pump that supplies and regulates a solution containing an aroma compound to the at least one conductive capillary of the vaporization unit; a high voltage unit in electrical contact with the conductive capillary of the vaporization unit for applying an electric charge to the solution and producing a vaporized aroma; and at least one gas flow controller connected to the gaseous inlet of the vaporization unit to create a gaseous stream of the vaporized aroma. In some embodiments, (i) the high voltage unit applies a DC voltage ranging between 2 kV to 6 kV, (ii) the high voltage unit applies an AC voltage frequency of between 0 Hz to 10 kHz, (iii) the pump provides a liquid flow rate of between 5 nL per minute to 1 mL per minute, (iv) the gas flow controller provides a gaseous flow rate of between 100 mL per minute to 1 L per minute, or (v) a combination of one or more of (i) to (iv). In other embodiments, the device: (i) is capable of using a carrier gas that does not generate ozone, (ii) uses a carrier gas that does not react with the aroma compound, (iii) has an aroma admittance exceeding 100 nS, (iv) provides an ultimate vapor concentration that is calculable from the at least one flow controller and the at least one pump set rates, (v) delivers aroma compound mixtures in ratios independent of headspace equilibrium or evaporation rates, (vi) programmatically delivers instantaneous stimulus presentation at precise intervals, (vii) dynamically alters vapor concentrations programmatically via the at least one pump, (viii) dynamically alters vapor concentrations programmatically via the at least one flow controller, (ix) programmatically changes odorant's presentation cone dynamically, (x) programmatically synchronizes odorant presentation with auditory or visual stimuli, or (xi) a combination of one or more of (i) to (x). A system including the device, one or more fragrances and a vapor sensor is also provided.

The invention further provides a method for vapor generation of an aroma compound which involves the step of introducing a solution containing an aroma compound into the device of the invention to vaporize the aroma compound so that, in some embodiments, the vaporized aroma compound is delivered to a subject for psychophysical evaluation. In certain embodiments, the for psychophysical evaluation includes (i) a dose response determination, (ii) a threshold determination, (iii) a malodor coverage evaluation, (iv) an adaptation/cross-adaptation evaluation, (v) an odor temporal processing measurement, (vi) a substantivity evaluation, (vii) a diffusivity evaluation, (viii) a subjective or objective response from the subject, (ix) a conscious or subconscious response from the subject, or (x) a combination of one or more of (i) to (ix). In other embodiments, the subject carrying out the evaluation is (i) a person with no prior training for evaluation, (ii) a person with prior training for evaluation, (iii) a person with expert knowledge of evaluation, or (iv) a person participating in a demonstration.

### BRIEF DESCRIPTION OF THE DRAWINGS

FIG. 1 shows a schematic representation of an electrospray ionization olfactometer device of this invention.

FIG. 2 shows a schematic section through a nozzle containing and inner nozzle and outer nozzle arrangement.

FIG. 3 shows a schematic representation of an electrospray ionization olfactometer device of this invention, which can deliver a combination or mixture of fragrances.

### DETAILED DESCRIPTION OF THE INVENTION

This invention provides an olfactometer system, device and method, which uses electrospray ionization to disperse aroma compounds into droplets without the use of a heating element. For the purposes of this invention, the terms fragrance, aroma and odor are used interchangeably. As known in the art, an olfactometer is a device used for the presentation of an odorized air stream to a subject(s). The term “electrospray” used herein, unless otherwise indicated, refers to an electrostatic liquid spray that operates below the potential for a corona electric discharge and disperses droplets of liquid from ligands formed at the tip of one or more so called Taylor cones, as well known to one skilled in the art. Alternatively stated, electrospray ionization vaporizes liquids via the presence of a strong electric field. The liquid is charged in a conductive capillary and as it emerges from the tip, the electric field's voltage potential overcomes the surface tension of the liquid causing the molecules of the liquid to repel each other and become small droplets. Because the phase change of aroma compounds occurs sans heat, there is no condensation, oxidation, or other detrimental reactions occurring. This technique maintains constant vapor concentration even at low pump rates, ensuring that aroma compounds with low volatility are able to be reliably vaporized and tested.

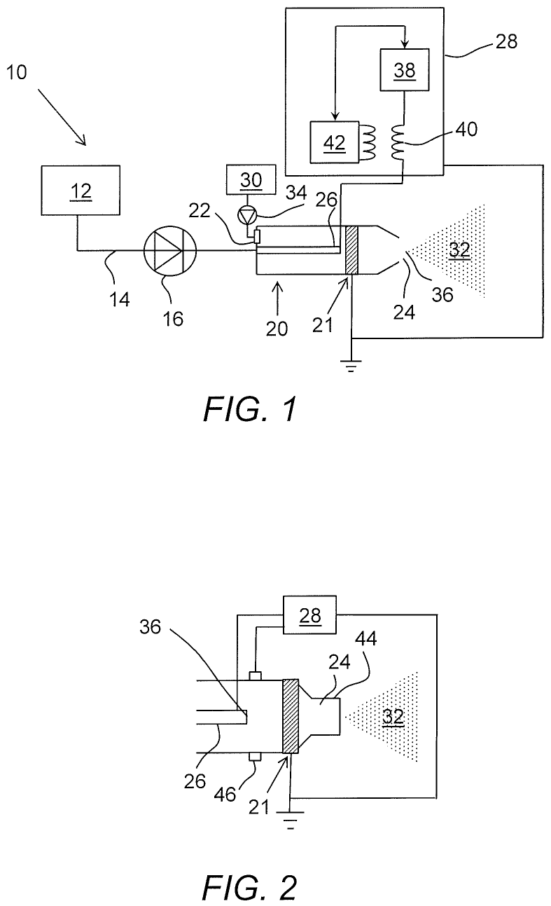

FIG. 1 shows a schematic of the electrospray ionization olfactometer device of the invention 10, which includes a single fragrance reservoir 12 for storing a fragrance or odor compound. Reservoir 12 can be integrated or releasably associated with the device and exchanged later, when empty, for a reservoir containing the same or a different fragrance. The fragrance is supplied via a supply line 14 to a pump 16. The pump 16 transports the fragrance to the vaporization unit 20, which includes a gaseous inlet and a gaseous outlet 24. Transport of the fragrance through the vaporization unit 20 is via a conductive capillary 26. In the vaporization unit, the fragrance is converted into an aerosol through application of an electric charge A high-voltage unit 28 in electrical contact with the conductive capillary 26 applies the electric charge to the fragrance, which exits the device as charged droplets 32 into the surrounding atmosphere.

With the device of this invention, a sample solution containing an aroma compound, fragrance or odor flows to a conductive capillary and a high voltage is applied. The solution in the capillary becomes charged. The highly charged droplets, with the same polarity and emerging from a capillary tip of the vaporization unit, repel each other and form an extremely fine mist which is visible only within a small distance from the vaporization unit. In this respect, the electrospray device and method can be used to controllably dispense very small quantities of a fragrance or aroma compound. The particle size of the generated electrospray is advantageously very uniform and, more importantly, very small, e.g., smaller than a micrometer. In this respect, the device can take the form of a microscale electrospray emitter. See U.S. Pat. No. 7,829,847, incorporated herein by reference.

In accordance with this invention, the fragrance is fed into the vaporization unit via a pump. In this respect, the pump provides the driving force to push the fluid through the conductive capillary of the vaporization unit and out though a nozzle located at the opposing end of the vaporization unit. In some embodiments, the pump operates with a liquid flow rate in the range of between 5 nL/minute to 1 mL/minute, or preferably, 5 nL/minute to 500 μL/minute, or more preferably 5 nL/minute to 1 μL/minute, or most preferably 5 nL/minute to 500 nL/minute. Pressure-based, electrokinetics-based, and displacement-based pumping techniques can be used. As a general matter, pressure pumping generates a proscribed pressure difference at the two ends of a pipe. See, e.g., Chien et al. (2001) Fresenius J. Anal. Chem. 371:106-11. Electrokinetic pumping techniques generally include electro-osmotic, electrophoretic, electro-wetting, and electrohydrodynamic (EHD) pumping, each of which operates on different principles than pressure and displacement pumping. For a general treatment of some types of electrokinetic pumping, see, e.g., Bousse, et al. (2000) Annu. Rev. Biophys. Biomol. Struct. 29:155-81. Displacement pumping generates a proscribed flow rate directly, typically by pushing a piston or other boundary against a volume of liquid. The change in volume generated by motion of the solid boundary, therefore, is the flow rate generated by the pump.

In particular embodiments of this invention, the pump is highly sensitive and allows for pump rates in the nL/minute range. In accordance with this embodiment, the pump used is a displacement pump, in particular, a syringe pump. A syringe pump is typically composed of a motor connected to, for example, a worm gear that pushes the plunger of a syringe, causing liquid to flow out of the syringe tip. Combined with a small inner diameter syringe, complete e-spray is achievable at low flow rates. The syringe is often coupled to whatever device or instrument requires the flow. Syringe pumps designed for low flow rates are commercially available. Syringe pumps can use stepper motors, which dispense predefined aliquots of liquid as pulsatile flows. However, when a linear, or non-pulsatile liquid flow rate is desired, a servomotor can be used. See, U.S. Pat. No. 8,021,130, incorporated herein by reference in its entirety. Preferably the syringe pump unit includes a 10 μl to 50 μl gas-tight syringe (e.g., a 10, 20 or 50 μl syringe) such that the syringe pump can be set to any substantially constant flow rate between about 5 nL/minute and 50 nL/minute. The pump is desirably computer controlled enabling starting/stopping programmatically, as well as altering the pumping speed (and thereby the vapor concentration) on the fly.

The fragrance flows from the pump to a conductive capillary of the vaporization unit. The term “capillary” refers to any sleeve, conduit, transport device, dispenser, nozzle, hose, pipe, pipette, port, orifice, connector, tube, coupling, container, housing, structure or apparatus that may be used to receive or transport a fluid sample. Desirably, the conductive capillary has an inner diameter between about 25 μm and 1000 μm or more desirably 25 μm and 100 μm and is electrically connected to a high voltage source by any conventional means, e.g., by leads or an alligator clip clipped onto the capillary.

In one embodiment, the capillary is produced from an electrically conductive material. Suitable electrically conductive materials include, e.g., copper, silver or stainless steel. In another embodiment, the capillary is produced from a nonconducting material that includes an electrically conductive layer or coating and a metallized tip. See, U.S. Pat. No. 5,630,925, incorporated herein by reference. The electrically conductive layer or coating may be formed by means of painting a metallic paint solution or vapor deposition of a metal such as aluminum or a noble metal such as gold onto the outside surface of the capillary so that, as long as there is some moisture or other liquid at the tip of the capillary, the layer or coating is electrically connected to the fragrance inside the capillary. In a preferred embodiment, the capillary is composed of a nonconducting material (e.g., a plastic such as PC, PMMA, PUR, PI, etc.) and an electrode in wire form arranged in the capillary, wherein the capillary and/or the electrode are formed and/or arranged in such a way that, during operation of the device, the electrode is constantly wetted and no oxygen from the ambient air has contact with the surface of the electrode. In this respect, ozone production is inhibited or minimized. See US 2009/0314850.

The high-voltage unit 28 of device 10 supplies AC voltage superimposed on the DC voltage. As illustrated in FIG. 1, DC voltage Vd is applied to capillary 26 from a high voltage DC power source 38 through a transformer 40. Alternatively, a capacitor is used instead of a transformer. Further, AC voltage Va is applied to the capillary 26 from an AC power source 42 through transformer 40, at the same time. That is, Voltage Va+Vd obtained by the DC voltage superimposed on AC voltage is applied to the nozzle. See, e.g., U.S. Pat. No. 6,737,640, incorporated herein by reference. AC-modulated DC field is created in the vicinity of capillary 26 by this superimposed voltage. By way of illustration, DC voltage is provided at 4 kV, and an AC sine wave of +/−1 kV is superimposed thereon, which results in a voltage oscillating between 3 kV and 5 kV. In some embodiments, a positive voltage is applied. In other embodiments, the high-voltage unit applies a DC voltage ranging between 2 kV to 6 kV, or more preferably between 2.5 kV and 5 kV. In still further embodiments, the high voltage unit applies an AC voltage frequency of between 0 Hz to 10 kHz, or more preferably between 10 Hz and 1 kHz. In particular embodiments, ground target 21 is also included as a common ground linked to high-voltage unit 28. In accordance with this embodiment, the distance between conductive capillary 26 and ground target 21 is preferably less than 2.5 cm and more preferably less than about 1.3 cm. The power source supplying the high voltage is preferably computer-controlled and is able to set/vary voltage rates and frequencies programmatically.

As the aroma compounds pass through the capillary, a mist of highly charged droplets with the same polarity as the capillary voltage is generated. The application of a carrier gas, which shears around the eluted sample solution, provides a higher sample flow rate and facilitates transmission of the vaporized fragrance through the nozzle. Accordingly, as illustrated in FIG. 1, a carrier gas is introduced into gaseous inlet 22 of vaporization unit 20 and leaves vaporization unit 20 through gaseous outlet 24 as a jet of gas surrounding the vaporized fragrance to form ultra-fine charged droplets 32.

As shown in FIG. 1, the gas flow from gas source 30 is controlled by gas flow controller 34 connected to the gaseous inlet 22. Desirably, the gas flow controller operates at a gaseous flow rate of between 100 mL/minute to 1 L/minute, or more preferably between 500 mL/minute to 1 L/min. While any suitable gas can be used as a carrier gas including, but not limited to dry air, argon, neon, oxygen or nitrogen, in certain embodiments, the carrier gas does not generate ozone and/or does not react with the fragrance, e.g., via oxidation or other chemical reaction.

As illustrated in FIG. 2, gaseous outlet 24 can include an outer nozzle 44, inside which nozzle 36 is arranged thereby facilitating transport of aroma compounds atomized by nozzle 36 out of nozzle 44 into the ambience. See US 2009/0314850. In some embodiments, the device optionally includes a second electrode 46, which may be arranged on outer nozzle 44 as, e.g., an annular electrode. Alternatively, electrode 46 may be formed as a grid with partial coverage of the opening of nozzle 44.

As appreciated by persons skilled in the art, the pump, gas flow controller and high-voltage unit functions can be implemented individually or integrated using a control unit including hardware (e.g., a personal computer), firmware (e.g., application-specific), software, or combinations thereof. A computer-based control unit can be a general-purpose computer that includes a memory for storing computer program instructions for carrying out processing and control operations. The computer can also include a disk drive, a compact disk drive, or other suitable component for reading instructions contained on a computer-readable medium for carrying out such operations. In addition to output peripherals such as a display and printer, the computer can contain input peripherals such as a mouse, touch screen, keyboard, barcode scanner, light pen, or other suitable component known to persons skilled in the art for enabling a user to input information into the computer.

In addition to control over the fragrance injection rate, mass flow controllers (MFCs) can be used to control the gaseous streams interacting with the electrospray and the presentation of the stimuli. One MFC per device can control the gaseous inlet of pure inert carrier gas (e.g., nitrogen) responsible for the initial vapor phase concentration in conjunction with the pump. At the gaseous outlet of the vaporization unit, a second MFC offers a diluting factor by removing a set amount of flow from the original stream. In this respect, the present device allows for the production of vapor concentrations well below other olfactometer devices and methods. The resultant flow is finally combined with humidified air provided by another MFC and is presented to a subject. In certain embodiments, each MFC is controlled by a computer control unit thereby allowing for dynamic control of flow rates.

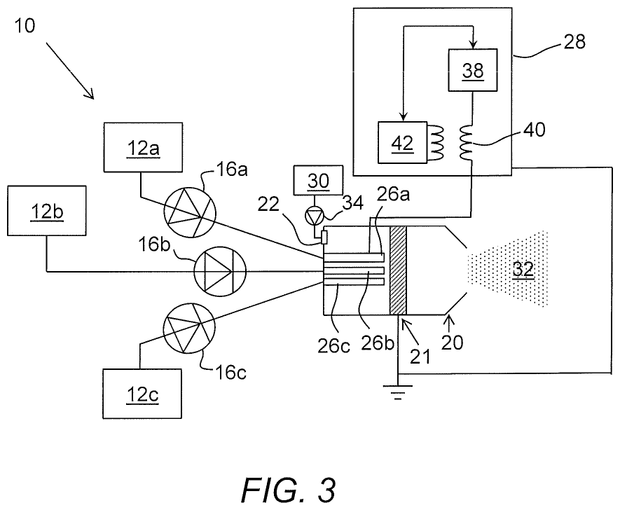

In certain instances, a number of different fragrances may be released simultaneously, whereby the intended olfactory effect is attained only by the superposition of the scents. This requires that the fragrances are in harmony with each other and that the individual quantities of the fragrances are exactly matched to each other. In this respect, FIG. 3 illustrates device 10 contain more than one fragrance reservoir 12a,12b,12c, more than one pump 16a,16b,16c to supply and regulate different aroma compounds, and more than one conductive capillary 26a,26b,26c. In certain embodiments, the device includes one vaporization unit. In other embodiments, the device includes more than one vaporization unit, each associated with its own fragrance reservoir and pump. In yet other embodiments, the device has a central pumping unit connected to a vaporization unit, wherein the pump can have several supply lines, with each of the supply lines supplying a different fragrance. The supply lines can have different cross-sections so that different quantities of fragrance can be suctioned by the suction effect of the pump either directly into the pump or supplied to an upstream mixing chamber, where the fragrances are then mixed and delivered as a combination of fragrances to the pump.

In addition to simultaneous release, the device can deliver several different fragrances with a time delay. For example, a particular “scent history” can be narrated by delivering individual fragrances with a time offset, without superimposing the individual fragrances.

In use, the device can provide mixtures of compounds that are vaporized according to the proportion they exist in the liquid state, allowing the creation of novel mixtures in the vapor phase that cannot be achieved through headspace or heating techniques. By way of illustration, Table 1 provides a mixture of fragrances for GERNIOL COEUR that can be created using the device of this invention.

TABLE 1

| | | | | E-Spray @ 0.4 |
|---|---|---|---|---|
| | GC peak | HS @ 1% | | μL/min in |
| Peak Name | Weight % | DEP | % HS | 10 LPM air |
| Geraniol | 57.1 | 1.61 μg/L | 43.3 | 57.9% |
| Citronellol | 27.0 | 1.69 μg/L | 45.5 | 27.6% |
| Nerol | 14.3 | 0.42 μg/L | 11.2 | 14.5% |

GC, gas chromatography; HS, head space; LMP, liters per minute.

Additional fragrances that can be used or combined, include, but are not limited to, allyl amyl glycolate, D-limonene, linalool synthetic, amyl acetate, ethylvanillin, and Galaxolide White.

Whether using a single fragrance or a mixture of fragrances, preferably the device of the invention includes the use of solenoids in the form of pinch valves, e.g., 2-way, 3-way or 4-way valves, to control release of the fragrances. More specifically, the pinch valves are computer controlled on/off switches, which direct flow through the capillaries. If a vapor stream is presented to one of three end ports, these valves can dynamically change which of the three contains the stream. Conventional olfactometers do not exhibit this level of stimulus presentation. Similarly, the pinch valves can control the on/off presentation of the overall odor stimulus. The valves can be connected to a MFC attached to a vacuum. In normal operating mode, the valves allow the passage of the vapor stream directly to the vacuum and no smell is detected at the end ports. When the valve is activated, either via computer or manual actuator, the valve begins to transmit ambient air to the vacuum and allows the passage of the vapor stream. The result is an instantaneous presentation of odor and instantaneous elimination. This presentation can be coupled with computer controlled visual and/or auditory stimulus. In this respect, the design of this device offers a control of stimulus presentation unparalleled with known devices.

To facilitate identification and dispensing, fragrance reservoirs can be coded, with the code being read by a suitable reading unit when the reservoir is attached to the device. The reading unit transmits the read information to the computer control unit, which controls the dose of the newly added fragrance reservoir based on this information. In this way, the dose can be adapted to the fragrances of the already existing fragrance reservoirs. In the device, the computer control unit can also react to a change in the fragrance supply, for example, when a fragrance reservoir is empty, by either providing a corresponding signal and/or modifying the quantities of the various fragrances.

When in use, the device desirably provides one or more of the following functions: vapor generation of any aroma chemical or mixture of aroma chemicals with admittance exceeding 100 nS; ultimate vapor concentration that is calculable from the at least one flow controller and the at least one pump set rates; ability to deliver aroma compound mixtures in ratios independent of headspace equilibrium or evaporation rates; ability to programmatically deliver instantaneous stimulus presentation at precise intervals; ability to dynamically alter vapor concentrations programmatically via the at least one pump and/or the at least one gaseous flow controller; ability to programmatically change odorant's presentation cone dynamically; and ability to programmatically synchronize odorant presentation with auditory or visual stimuli.

This invention also provides a method for vaporizing aroma compounds for psychophysical experimentation. The method involves using the electrospray ionization olfactometer device of this invention for vapor generation and optionally presentation of aroma compounds to at least one subject for psychophysical experimentation. For the purposes of this invention, psychophysical experimentation refers to the evaluation of one or more of the following characteristics of a single aroma compound or mixture of aroma compounds: dose Response determination; threshold determination; malodor coverage evaluation; adaptation/cross-adaptation evaluation; odor temporal processing measurements; substantivity evaluation; and diffusivity evaluation. In addition to the above, other subjective and/or objective responses and/or conscious and/or subconscious responses from the at least one subject can be obtained.

Subjects carrying out the method of this invention include, but are not limited to, a person or persons with no prior training for evaluation; a person or persons with prior training for evaluation; a person or persons with expert knowledge of evaluation; a person or persons participating in a demonstration and a person or persons participating in training.

In some embodiments, the device is used in combination with a vapor sensor or electronic nose to monitor and/or calibrate the olfactometer device. Therefore, this invention also provides a system containing the electrospray ionization olfactometer device, one or more fragrances and a vapor sensor. Various types of vapor sensors can be used in monitoring and calibrating an olfactometer device. Electronic noses, which are capable of analyzing complex odors and vapors, are discussed, e.g., in Baletz et al. (1998) IEEE Spectrum, pp. 36-38; Kaplan & Braham (1998) IEEE Spectrum, pp. 22-34. Electronic noses work by comparing process signals from a sensor array with known patterns stored in a data base. Various types of sensor arrays which are possible include conductive polymer sensors (U.S. Pat. Nos. 5,801,297; 5,145,645; 4,911,892; and 5,756,879), metal oxide conductivity sensors (U.S. Pat. No. 5,777,207), quartz resonator type sensors (U.S. Pat. No. 5,177,994), colorimetric sensors (U.S. Pat. Nos. 6,495,102; 7,261,857) polymerdielectric sensor (capacitor), fluorescent optical sensor, etc. The type of sensor will determine the key features: number of sensor elements, detector sensitivity (threshold and response curve), stability, reproducibility, response time and refresh time. In some embodiments, the device self-calibrates before each new test or on command. In other embodiments, the sensor signal causes a visual or audible signaling device to show that the vapor was or was not present at the test location when a dispensing step was performed. In other embodiments, the vapor sensor or electronic nose is connected or in electronic communication with the olfactometer device in a closed feedback loop in order to control the concentration and/or composition of the delivered fragrance. In this respect, the system output can be taken into consideration thereby enabling the system to adjust its performance to meet a desired output.

Unlike devices based upon heat or evaporation, where the most volatile components are dispersed at a faster rate than less volatile components, the electrospray ionization olfactometer device of this invention can dispense all components in a mixture at an equal rate. Therefore, the design of the olfactometer device of this invention allows for superior stimulus control as compared to conventional olfactometers. The olfactometer of this invention can be used to dispense aromas, fragrances and vapors from a computer with an attached or separate aroma box or headset; radio or television set; automobile, cell phone or telephone; home appliance such as stand-alone air freshener, personal items such as eyeglasses, broach or pocket unit; or a medical instrument or device.

The invention is described in greater detail by the following non-limiting examples.

### Example 1: Compact/Distributable Olfactometer

The device is produced as a compact or distributable olfactometer, wherein all the components necessary to produce vaporized aroma fragrance are assembled into a portable unit. Such a device can be used for in-house or third party sensory testing and can be produced for purchase, lease or rent.

### Example 2: Miniaturized/MEMS Chip Device

For fragrance delivery at the micro-scale level, a miniaturized/microelectromechanical sensor (MEMS) chip device can be produced. Such a device has a wide range of end-uses due to its size and can be used in any application where fragrance delivery is desired. By way of illustration, such a device can be used in a space-restricted application such as in smart phone or tablet scent transmission. In this respect, a digital signature can be sent between users, wherein a scent is recreated on the receiving device. A miniaturized/MEMS chip device can also be integrated into augmented/virtual reality setups to enhance multisensory experience. In certain embodiments, the device can be provided as a removable cartridge.

### Example 3: Commercial Kiosk

The device described herein is produced as a commercial kiosk, wherein consumers can navigate a software program on a touch screen and select fragrances to smell. Due to the compact nature of the device and the relatively small amount of chemical necessary to generate odor, one could store a large quantity of different fragrances. Users can choose a fragrance not by blotter evaluation and walking around a store, but by dedicated instruments, thereby reducing blotter/trash waste.

### Example 4: Fragrance Bank

The device can take the form of a fragrance bank, where perfumers are able to recall fragrance accords stored in a robotic array, much as the commercial kiosk described in Example 3. This could include accords generated from measurement of the headspace above naturally scented or aromatic items (e.g., flowers, baked goods, or fragrance headspace measured in application).

### Example 5: Formula Creator

The device described herein can store fragrance ingredients that can be accessed independently of one another. A perfumer can formulate a fragrance dynamically, by adding components at specified concentrations to an air stream. In this device, the number of fragrance ingredients is only limited by size of the device.

What is claimed is:

1. A device for vaporizing aroma compounds without heat, comprising:
a) at least one vaporization unit, which includes at least one conductive capillary, at least one gaseous inlet, and at least one gaseous outlet;
b) at least one pump that supplies and regulates a solution containing an aroma compound to the at least one conductive capillary of the vaporization unit;
c) a high voltage unit in electrical contact with the conductive capillary of the vaporization unit for applying an electric charge to the solution and producing a vaporized aroma; and
d) at least one gas flow controller connected to the gaseous inlet of the vaporization unit to create a gaseous stream of the vaporized aroma.

2. The device of claim 1, wherein
(i) the high voltage unit applies a DC voltage ranging between 2 kV to 6 kV,
(ii) the high voltage unit applies an AC voltage frequency of between 0 Hz to 10 kHz,
(iii) the pump provides a liquid flow rate of between 5 nL per minute to 1 mL per minute,
(iv) the gas flow controller provides a gaseous flow rate of between 100 mL per minute to 1 L per minute, or
(v) a combination of one or more of (i) to (iv).

3. The device of claim 1, wherein said device
(i) is capable of using a carrier gas that does not generate ozone,
(ii) uses a carrier gas that does not react with the aroma compound,
(iii) has an aroma admittance exceeding 100 nS,
(iv) provides an ultimate vapor concentration that is calculable from the at least one flow controller and the at least one pump set rates,
(v) delivers aroma compound mixtures in ratios independent of headspace equilibrium or evaporation rates,
(vi) programmatically delivers instantaneous stimulus presentation at precise intervals,
(vii) dynamically alters vapor concentrations programmatically via the at least one pump,
(viii) dynamically alters vapor concentrations programmatically via the at least one flow controller,
(ix) programmatically changes odorant's presentation cone dynamically,
(x) programmatically synchronizes odorant presentation with auditory or visual stimuli, or
(xi) a combination of one or more of (i) to (x).

4. A method for vapor generation of an aroma compound comprising introducing a solution containing an aroma compound into the device of claim 1 to vaporize the aroma compound.

5. The method of claim 4, wherein the vaporized aroma compound is delivered to a subject for psychophysical evaluation.

6. The method of claim 5, wherein the psychophysical evaluation comprises:
(i) a dose response determination,
(ii) a threshold determination,
(iii) a malodor coverage evaluation,
(iv) an adaptation/cross-adaptation evaluation,
(v) an odor temporal processing measurement,
(vi) a substantivity evaluation,
(vii) a diffusivity evaluation,
(viii) a subjective or objective response from the subject,
(ix) a conscious or subconscious response from the subject, or
(x) a combination of one or more of (i) to (ix).

7. The method of claim 5, wherein the subject is:
(i) a person with no prior training for evaluation,
(ii) a person with prior training for evaluation,
(iii) a person with expert knowledge of evaluation, or
(iv) a person participating in a demonstration.

8. A system comprising the device of claim 1, one or more fragrances and a vapor sensor.

9. The system of claim 8, wherein the vapor sensor is connected to the device in a closed feedback loop to control the fragrance concentration and/or composition.
